(19) United States
(12) Patent Application Publication
(10) Pub. No.: US 2010/0055138 A1
(43) Pub. Date: Mar. 4, 2010

(54) METHODS AND TOPICAL FORMULATIONS COMPRISING COLLOIDAL METAL FOR TREATING OR PREVENTING SKIN CONDITIONS

(76) Inventors: JOEL MARGULIES, LASCASSAS, TN (US); TERRY JACOBS, MARINA DEL REY, CA (US)

Correspondence Address: BLAKELY SOKOLOFF TAYLOR & ZAFMAN LLP, 1279 OAKMEAD PARKWAY, SUNNYVALE, CA, 94085-4040, US

(21) Appl. No.: 12/488,502

(22) Filed: Jun. 19, 2009

Related U.S. Application Data
(63) Continuation of application No. PCT/US2007/088218, filed on Dec. 19, 2007.
(60) Provisional application No. 60/875,998, filed on Dec. 20, 2006.

(51) Int. Cl. A61K 8/14; A61K 33/24; A61K 39/00; A61K 8/66; A61K 8/64; A61K 8/60; A61K 8/73; A61Q 19/00

(52) U.S. Cl. 424/401; 424/649; 424/193.1; 424/94.1; 514/6; 514/23; 514/54

(57) ABSTRACT

In preferred embodiments, the present invention relates to compositions comprising colloidal metals and/or metals for the treatment and prevention of skin conditions and/or diseases. More specifically, the disclosed metal containing compositions are useful as antioxidants, anti-aging agents, anti-wrinkle agents, anti-peroxidation agents, antimicrobial agents, anti-inflammatory agents, pain-relieving agents, wound recovery agents, sun-screens, sunblocks, and integument and skin-supporting agents when applied to the skin/integument, or administered generally to an animal or human body.

### CROSS-REFERENCE TO RELATED APPLICATION

This application claims the benefit of International Patent Application No. PCT/US2007/088218, filed Dec. 19, 2007, which claims the benefit of U.S. Patent Application No. 60/875,998, filed Dec. 20, 2006.

### BACKGROUND OF THE INVENTION

1. Field of the Invention

In preferred embodiments, the present invention relates to compositions comprising colloidal metals and/or metals for the treatment and prevention of skin conditions and/or diseases. More specifically, the disclosed metal containing compositions are useful as antioxidants, anti-aging agents, anti-wrinkle agents, anti-peroxidation agents, antimicrobial agents, anti-inflammatory agents, pain-relieving agents, wound recovery agents, sun-screens, sunblocks, and integument and skin-supporting agents when applied to the skin/integument, or administered generally to an animal or human body.

2. Description of the Related Art

The integumentary system is the largest organ system of an animal by surface area. This system includes skin, hair, feathers, scales, nails, and sweat glands and their products (sweat and mucus). The skin comprises three primary layers: the epidermis, dermis, and subcutaneous layer. For mammals, including humans, the skin provides a water-resistant and nearly impenetrable barrier against invasion by microorganisms.

Many conditions and diseases can affect the skin's integrity, health, and appearance. These include but are not limited to, acne, psoriasis, rosacea, inflammation, sunburn, and infection. For example, acne is a group of diseases whose initial pathology is the comedo and includes acne vulgaris, neonatal acne, infantile acne, and pomade acne. Acne affects between 40 million and 50 million individuals in the United States. Although acne mainly affects adolescents, some individuals more than the age of 25 years, about 54% of women and 40% of men, experience some degree of facial acne. (Cordain et al., Arch Dematol 2002; 138: 1584-1590.)

There are many causes of acne including hormonal activity, bacteria, age, and stress. However, acne can ultimately be attributed to blocked hair follicles (comedones). Besides hormonally induced blockage of hair follicle orifices by body fats, a major cause of acne is the development of tissue-damaging free fatty acids and enzymes by bacteria such as, for example, the propionibacterium.

Other examples of conditions that can affect the skin's integrity, health, and appearance are infections from physical trauma to the skin such as bruising, cuts, larcerations, in-grown nails, and sores. Although nature has equipped most organisms with immune systems capable of combating infection and assisting wound healing and recovery, oftentimes the healing process can be slow and painful, as well as, lead to aesthetically undesirable side-effects such as open, wet, pus-releasing, and/or bleeding wounds. Moreover, for some individuals such as those with diabetes, problems with chronic wounds, indolent, or nonhealing wounds may arise.

Apart from infection and disease, other external environmental factors can contribute to the degradation of the skin and integument. For example, aging is a natural progression of life for every living organism. Aging is usually thought of as the gradual deterioration of an organism's physiological systems over time. The manner and rate of this progression is affected by a myriad of factors including one's genetics, lifestyle, and environment. Skin-aging is a phenomenon that reflects both physiological aging and environmental aging due to exposure to external factors such as ultraviolet radiation from sunlight (photoaging). Skin-aging can be manifested in many forms including poorly hydrated skin, skin discoloration, wrinkles, and/or skin damage resulting in cancerous tissue such as melanoma. Although skin-aging and aging in general are inevitable for every living organism, the exposure to external factors can accelerate or exacerbate this process. Examples of additional external factors affecting skin/integument health include exposure to tobacco, airborne particulates, gases, cleaning agents, fertilizers, alcohol, and/or sunlight.

Sunlight, particularly ultraviolet radiation, contributes to this process through photoaging. The skin contains an elaborate network of elastin fibers that is responsible for maintaining its elastic properties. With excessive exposure to sunlight the elastic fiber system can become hyperplastic, disorganized and ultimately disrupted. This process can contribute to wrinkling, discoloration, and laxity of the skin in the exposed areas of the body. As new fibroblasts, endothelial cells and keratinocytes form, the skin can repair itself. However, the skin becomes less able to do so as it ages.

Moreover, ultraviolet radiation has been found to induce increased production of collagenase/matrix metal loproteinases (MMPs), Fischer, et al., J. Invest. Dermatol. 2001 August; 1 17(2):219-26. Collagenase and MMPs are enzymes that breakdown and degrade collagen in the skin. MMPs refer to any protease of the family of MMPs which are involved in the degradation of connective tissues, such as collagen, elastin, fibronectin, laminin, and other components of the extracellular matrix. This breakdown from both collagenase and MMPs can lead to accelerated aging, wrinkling, and cancerous tissues.

Additionally, without wishing to be bound by any theory, it is believed that exposing skin to ultraviolet rays initiates and increases lipid peroxidation in the skin. Lipid peroxidation in the skin refers to a process where free radicals cause damage to vital skin components such as collagen and elastin. Collagen constitutes 90% of the skin's dermis and is distributed all over the dermis to give appropriate elasticity and strength to the skin. Lipid peroxidation can occur, for example, when photons of ultraviolet radiation generate free radicals in the skin. Inflammation, wrinkling, and roughening of the skin are some symptoms associated with skin damage from lipid peroxidation.

Because radical formation is a key factor in skin-aging, anti-oxidizing agents are particularly important in reducing and preventing lipid peroxidation. Antioxidants inhibit lipid peroxidation, which prevents and minimizes the damage contributing to skin-aging. Natural anti-oxidizing enzymes such as superoxide dismutase are effective antioxidants in mitigating and preventing the damage caused by free radicals. Specifically, superoxide dismutase converts free radical oxygen species (ROS) into water and oxygen molecules. However, problematically, natural superoxide dismutase levels decrease with aging.

Because of the many conditions and diseases, both external and internal, impacting the health of the skin and integument, there is a need for compositions that can provide multi-faceted protection and treatment and prevention of a variety of conditions that may affect the well-being of subject or patient. The present invention addresses this need by providing colloidal metal and/or metal flake compositions with anti-oxidizing, antigen, anti-aging, anti-wrinkling, anti-peroxidizing, antimicrobial, anti-inflammatory, pain-relieving, wound recovery, sun-screening, sun-blocking, integument and skin-supporting, and regenerative stimulating effect.

### SUMMARY OF THE INVENTION

The preferred embodiments of the present invention have several features, no single one of which is solely responsible for their desirable attributes. Without limiting the scope of this invention, its more prominent features will now be discussed briefly. However, not all of the following features are necessary to achieve the advantages of the invention. Therefore, none of the following features should be viewed as limiting. After considering this discussion, and particularly after reading the section entitled “Detailed Description of the Preferred Embodiments,” one will understand how the features of the preferred embodiments provide advantages over prior art.

In one aspect of the invention, metal containing compositions are provided. A composition, according to the present invention, preferably comprises a colloidal metal and suitable carrier. In some embodiments, the colloidal metal is colloidal gold. Preferably, the colloidal metal has a particle size from about 1-200 nm. In additional embodiments, the compositions comprise colloidal metal and metal flakes. In some embodiments, the metal flakes have a particle size from about 1-5 mm. In further embodiments, the colloidal metal comprises from about 0.001-25 percent by weight of the composition. In still further embodiments, the metal flakes or colloidal metal is selected from the group consisting of gold, silver, platinum, palladium, zinc, and copper. In other embodiments, the metal flakes or colloidal metal comprises a metal alloy. In further embodiments, the colloidal metal is conjugated to a molecule. In some embodiments, the molecule is selected from the group consisting of peptides, carbohydrates, enzymes, proteins, antigens, hormones, or polysaccharides. In further embodiments, the conjugated colloidal metal has a particle size from about 1 to 250 nm.

In another aspect of the invention, the suitable carrier for the described composition is adapted for topical, oral, mucosal, sublingual, enteral, and/or parenteral administration to a subject. In another embodiment, the suitable carrier may comprise of at least one emollient. In further embodiments, the suitable carrier may be a skin penetrating carrier. In preferred embodiments, the skin penetrating carrier may be DMSO, liposomes, lipophilic solvents, lecithin, transcutol, melatonin, nanospheres, nanoshells, cerasomes, and/or rovisomes. In other embodiments, the skin-penetrating carrier comprises a hollow and solid lipid structure. In another embodiment, the skin-penetrating carrier comprises a nano-structure. In further embodiments, the suitable carrier is selected from the group consisting of a sublingual formulation, transdermal patch, lotion, ointment, paste, foam, emulsion, cream, serum, aerosol, spray, roll-on formulation, masque, polish, cleanser, moisturizer, pill, tablet, caplet, capsule, and gel-cap. In additional embodiments, the suitable carrier can be a lubricating formulation, water-based formulation, silicone-based formulation, petroleum-based formulation, natural-oil based formulation, and/or massage formulation.

In another aspect of the present invention, the composition further comprises at least one additional agent. In some embodiments, the said at least one additional agent can be selected from the group consisting of minerals, antimicrobial agents, antioxidants, antigens, analgesics, anti-inflammatory agents, sebum-reducing agents, hormones, enzymes, peptides, proteins, lipids, retinoids, vitamins, wound recovery agents, botanical extracts, MMP inhibitors, integument and skin-supporting components, or massage oils.

In some embodiments, the composition further comprises an antimicrobial agent selected from the group consisting of triclosan, povidone, iodine, proflavine, honey, hydrogen peroxide, clotrimazole, or sulfur.

In other embodiments, the composition further comprises an antioxidant selected from the group consisting of beta glucan, curcumin, carnosine, polyphenolics, superoxide dismutase (SOD), catalase, glutathione peroxidase, oligomeric proanthocyanidins, bioflavonoids, oligomeric procyanidolic complexes, leuco anthocyanin, anthocyanidin, alpha-lipoic acid, coenzyme Q-10, selenium, vitamin E, vitamin C, lycopene, tocotrienols, or glutathione.

In further embodiments, the composition further comprises an analgesic comprising an amine-containing local anesthetic. In other embodiments, the composition further comprises an antihistamine analgesic. In yet further embodiments, the composition further comprises an analgesic selected from the group consisting of paracetamol, NSAIDs, benzocaine, butamben picrate, dibucaine, dibucaine hydrochloride, dimethisoquin hydrochloride, dyclonine hydrochloride, lidocaine, lidocaine hydrochloride, pramoxine hydrochloride, tetracaine, tetracaine hydrochloride, alcohols and ketones, benzyl alcohol, camphor, combinations of camphor and phenol, camphorated metacresol, juniper tar, menthol, phenol, phenolate sodium, resorcinol, antihistamines, diphenylhydramine hydrochloride, tripelennamine hydrochloride, hydrocortisone preparations, hydrocortisone, hydrocortisone acetate, allyl isothiocyanate, ammonia solutions, methyl salicylate, turpentine oil, histamine dihydrochloride, methyl nicotinate, capsaicin, capsicum, or capsicum oleoresin.

In some embodiments, the composition according to the present invention further comprises anti-inflammatory agents selected from the group consisting of hyssop, licorice extract, aloe, salicylic acid, allantaoin, bisabolol, or fumaric acid.

In other embodiments, the composition further comprises sebum-reducing agent selected from the group consisting of azelaic acid, revivogen, or MK-386 (4,7-[beta]-dimethyl-4-aza-5a-cholestan-3-one).

In further embodiments, the composition further comprises a peptide selected from the group consisting of palmitoyl pentapeptide, ubiquitin, oligopeptide, neuropeptide Y, pentapeptide, hexapeptide, acetyl hexapeptide-3, palmitoyl pentapeptide 3, epidermal growth factor (Egf), copper and copper containing peptides, thrombin, or fibroblast growth factor (Fgf).

In other embodiments, the composition further comprises a lipid selected from the group consisting of glycerides, phospholipids, phosphatidylcholine, or lecithin. Additionally, in some embodiments, the composition further comprises a retinoid selected from the group consisting of tretinoin, retinol, rose hips, or 9-cis retinoic acid.

Additionally, some particular embodiments may further comprise vitamins wherein the said vitamin is selected from the group consisting of vitamin A, B1, B2, B3, B5, B6, B7, B9, B 12, C, Ester-C, D, E, F, or K. Other embodiments may comprise vitamin containing compounds such as rose hips.

Further embodiments provide for wound recovery agents selected from the group consisting of allantoin, beta glucan, geranium extract, azelaic acid, curcumin, fumaric acid, gamma linolenic acid, farsenol, or squalene.

In another embodiment, the present invention provides for compositions comprising MMP inhibitors selected from the group consisting of minimal-domain MMPs, simple hemopexin domain-containing MMPs, gelatin-binding MMPs, furin-activated MMPs, and vitronectin-like insert MMPs, type I transmembrane MMPs, glycosyl-phosphatidyl inosital (GPI)-linked MMPs, or type II transmembrane MMPs.

In further embodiments, the present invention provides for compositions comprising polysaccharides such as beta glucan or dextran.

Additionally, some embodiments comprise integument and integument and skin-supporting components selected from the group consisting of vitamin K, borage oil, flax seed, cod liver oil, black currant, alpha and beta hydroxy acids, grape seed, pycnogenol, rose hips, sunscreens, tea tree oil, acetyl glucosamine algae, collagen, elastin, copper PCA, dead sea minerals, glycerin, hormone creams, human growth factor, kinetin, lanolin, mineral oil, olive oil, oxygen, perfluorodecalin (Rejuvenox), soy lecithin phospholipids, hydrogen peroxide, triclosan, salicylic acid, papain, aloe vera, lavender oil, geranium oil, chamomile, calendula officinalis, squalane, magnesium oxide crystals, macademia nut oil, galactoarabinan, magnesium aluminum silicate, sweet almond oil, sesame oil, palmitoyl-pentapeptide-3, peptides, benzoic acid, butylene glycol, carbomer, phyllanthus emblica fruit extract, urea, centella asiatica extract, echinacea angustifoila (coneflower) extract, hydrolyzed wheat protein, propylene glycol, bearberry extract, licorice, carnosine, caffeine, cocoa butter, kukui nut, shea butter, mugwort extract (artemesia vulgaris), mango butter, plantago lanceolate leaf extract, xanthan gum, sodium lauryl sulfate, glycolic acid, lactic acid, malic acid, citric acid, aartaric acid, all-trans retinoic acid, allantoin, aloe barbadensis, aminobutyric acid, arbutin, arginine amino acid, azelaic acid, caffeic acid, carnosine, retin-A, ceramides, copper gluconate, superoxide eismutase, curcumin, cystosine, beta glucan, dehydroepiandrosterone, DHEA, dinitrochlorobenzene, dipotassium glycyrrhizinate, hydantoin, DMSO, elastin, Ester-C, bromelian, erythropoietin (EPO), evening primrose oil, farnesol, fumaric acid, GABA (gamma aminobutyric acid/gamma amino-butyric acid), gamma linolenic acid, geranium extract, glutathione, glycyrrhiza glabra glycyrrhetic acid, hesperidin, hyaluronic acid, N6-Furfuryladenine Hormone, kojic acid, Tissue Inhibitors Of Metalloproteinases Matrixyl (TIMPS), vitamin E, vitamin C, flavonoids, palmitoyl panax ginseng root extract, pantothenic acid, pycnogenol scavenger, phosphatidylcholine, resveratrol, retinol, silicone, soy extracts, squalene, sulfur, saw palmetto, tocotrienols, ubiquinone, coenzyme Q10, botox, botulinum toxin, EGF, IGF, calendula officinalis, immortelle, green tea extract, white tea, black tea, glucosamine, algae, yeast, magnesium, magnesium aspartate, glycerin, progesterone, estrogen, lavender, cucumber, DNA, panthenol, zinc gluconate, camellia oleifera leaf extract, or pomegranate.

In additional embodiments, the composition comprises at least one additional agent selected from the group consisting of base components, surfactants, thickening agents, gelling agents, stabilizing agents, emulsifying agents, dispersing agents, suspending agents, humectants, emollients, acidic or alkaline substances, buffering agents, anti-crystalline agents, lubricating agents, coloring agents, perfumes, excipients, foaming agents, diluents, fillers, binding agents, or preservatives.

In another aspect of the present invention, the described components of the composition are encapsulated by a protective membrane. In some embodiments, the protective membrane can be a liposome, nanosphere, rovisome, cerasome, or nanoshell. In another embodiment, the protective membrane encapsulates a portion of the components. In further embodiments, a protective liposomal membrane encapsulates a portion of the components.

In a further aspect of the present invention, methods of treating and/or preventing a skin/integument condition are provided. In some embodiments, these methods provide for administering to a patient in need thereof, an amount of the described composition, wherein the amount is sufficient to treat and/or prevent the skin/integument condition. In other embodiments, skin condition is selected from the group consisting of acne, premature aging, ultraviolet radiation damage, damage from oxidative stress, damage from ROS, or damage from dehydration. In further embodiments, the skin condition comprises skin damage from exposure to tobacco, airborne particulates, gases, cleaning agents, fertilizers, or alcohol. In other embodiments, the skin condition is selected from the group consisting of rosacea, psoriasis, or other dermatitis condition. In an additional aspect of the present invention, methods of administration provide for topical, oral, transdermal, mucosal, enteral, parenteral, and/or sublingual administration.

In another embodiment, the present invention provides compositions consisting essentially of colloidal metal, metal flakes, a suitable carrier, salicylic acid, sebum reducers, humectants, wound recovery agents, beta glucan, superoxide dismutase, retinols, vitamin A, vitamin B, vitamin C, vitamin E, trace colloidal minerals, peptides, benzyl peroxide, sulfur, ceramides, and lipids.

In another embodiment, the present invention also provides compositions consisting essentially of colloidal metal, metal flakes, a suitable carrier, retinoids, idebenone, argiriline, MMP inhibitors, ceramide, vitamin A, vitamin B, vitamin C, vitamin E, trace minerals, salicylic acid, sebum reducers, humectants, wound recovery agents, superoxide dismutase, beta glucan, peptides, and lipids.

In another aspect, the present invention provides compositions consisting essentially of colloidal metal, metal flakes, a suitable carrier, idebenone, peptides, MMP inhibitors, ceramides, vitamin A, vitamin B, vitamin C, vitamin E, trace colloidal minerals, salicylic acid, sebum reducers humectants, wound recovery agents, superoxide dismutase, beta glucan, and retinoids.

In a further aspect, the present invention provides a composition consisting essentially of colloidal metal, metal flakes, enzymes, hormones, inhibitors, nutrients, proteins, antigens, antibacterials, surfactants, dermal barrier transport agents, and a suitable encapsulating membrane carrier.

### DETAILED DESCRIPTION OF THE PREFERRED EMBODIMENT

As noted above, the compositions and methods of preferred embodiments of the present invention relate to compositions comprising colloidal metals and/or metal flakes for the treatment and prevention of skin and integument conditions.

Antimicrobial

An “antimicrobial agent” as used herein generally refers to a material that inhibits the growth, survival and/or transmission of infections caused by bacteria, viruses, yeast, molds and funguses. In certain embodiments, an antimicrobial material has sufficient activity to provide beneficial therapeutic effect.

Although numerous organic compounds are known to possess potent contact-mediated antimicrobial actions (e.g., alcohol), such compounds are poorly suited for long-term protection. In accordance with preferred aspects of the present invention, inorganic metal containing materials are used as an antimicrobial agent, such as metals having antimicrobial properties. Silver, gold, copper, zinc, tin, mercury, lead, iron, cobalt, nickel, manganese, arsenic, antimony, bismuth, barium, cadmium and chromium have been known for a long time as metals which exhibit antifungal, anti-algal and antibacterial activities. For example, silver has been widely used in the form of aqueous silver nitrate solution as bactericidal and/or disinfectant solutions. Additionally, colloidal copper and copper PCA are believed to exhibit strong anti-fungal and antibacterial properties. However, some of the above-mentioned antimicrobial metals have been found to be toxic for humans. Furthermore, some of the toxic metals also have various practical and regulatory limitations in methods of use, storage and disposal. Consequently, their use as antimicrobial agents has been limited.

Recently, applicant has found that relatively small concentrations of colloidal antimicrobial metals, gold in particular, when combined with a non-toxic, suitable carrier, is sufficient to provide desired antimicrobial activity without risk of toxic effects for humans.

Composite materials, comprising a colloidal antimicrobial metal in combination with a pharmaceutically suitable carrier are preferred in accordance with the present invention. Gold-based inorganic antimicrobial agents are most preferred. These inorganic antimicrobial agents cause less skin irritation and offer much improved longevity, when compared with typical volatile organic agents.

Anti-Inflammatory

Metals can provide anti-inflammatory relief that is beneficial for the treatment and prevention of many physiological conditions where inflammation can cause discomfort, pain, as well as unappealing or undesirable aesthetic effects. An “anti-inflammatory agent” as used herein generally refers to a material or substance that has sufficient biological activity to reduce inflammation. In certain embodiments, an anti-inflammatory agent has sufficient activity to provide a beneficial therapeutic effect.

In accordance with preferred aspects of the present invention, inorganic metal containing materials are used as anti-inflammatory agents. For example, gold, copper, and silver have been known as metals that exhibit anti-inflammatory effects. In particular, gold has been widely used in the form of gold salts, such as disodium aurothiomalate, for the treatment and prevention of inflammation in rheumatoid arthritic conditions.

In some embodiments, a colloidal anti-inflammatory metal, such as colloidal gold, is combined with a non-toxic, suitable carrier, sufficient to provide the desired anti-inflammatory activity without risk of toxic effects for humans.

Anti-Oxidizing/Anti-Peroxidizing

Antioxidants inhibit inter alia lipid peroxidation, which prevents or minimizes damage to physiological systems. Such damage can contribute to accelerated aging or formation of cancerous tissues. Natural anti-oxidizing enzymes such as superoxide dismutase are effective antioxidants in mitigating or preventing the damage caused by free radicals associated with oxidation and lipid peroxidation. But natural superoxide dismutase levels decrease with aging.

As a result of intensive investigation into preventing oxidative and peroxidative damage, the present inventors have found that metals, such as gold and copper, exhibit anti-oxidizing/peroxidizing properties. Without wishing to be bound by any theory, it is believed that in some embodiments of the present invention, colloidal gold is an effective catalyst for the elimination of free radicals such as ROS (See e.g., Esumi et al., Langmuir, 2004, 20, 2536-2538). Accordingly, the colloidal metal containing compositions provide anti-oxidizing benefits for the skin/integument, which prevent and/or minimize the effects of radical damage.

Colloidal Metal and/or Metal Flakes

According to the present invention, some embodiments provide for a composition comprising an antimicrobial colloidal metal dispersion. Preferably, the colloidal metal has a particle size ranging about 1-200 nanometers (nm). In some embodiments, the colloidal metal has a particle size less than about 100 angstroms. In some embodiments, the particle size is less than about 25 angstroms. In some preferred embodiments, the particle size is about 1.5-5 nm. In some embodiments, the particle size of the colloidal metal can affect the metal's antimicrobial, anti-inflammatory, and/or anti-oxidizing properties. In other embodiments, the colloidal metal comprises about 0.001 to 50 percent weight of composition. In further embodiments, the pH of the colloidal formulation is from about 4.5-6. Additionally, some formulations may exhibit coloration.

The preparation of colloidal metal can be by various methods. For example, methods for preparing colloidal gold include citrate reduction whereby gold nanoparticles are produced by reduction of gold(III) derivatives, such as HAuCl₄ in water, or the Burst-Schiffrin Method.

In some embodiments, the colloidal metal consists of particles or clusters of pure metal suspended in pure and de-mineralized water with no ionic or electric charge. In other embodiments, the metal particles are in constant motion, exhibiting Brownian motion. Without being bound to any theory, it is believed that this motion results in charged metal particles. Consequently, in some embodiments, the like-charged metal particles exert repulsive forces on each other, which contribute to uniform distribution.

In some embodiments, the colloidal metal may be conjugated to a molecule such as a protein. Metal conjugates may be prepared by preparing metal sols by generally known procedures. For example, gold sols can be produced by reducing tetrachloroauric acid. In the case of gold, the gold sols are loaded with a molecule of choice, e.g. antigen, polypeptides, enzymes, nucleic acids, polysaccharides, proteins, etc. Without wishing to be bound by any theory, it is believed that conjugated colloidal metal, for example, antigen conjugated colloidal metal, targets specific sites in the skin or integument such that the colloidal metal and other composition components of the present invention are brought into the vicinity or contact with specific sites.

In some embodiments, the compositions of this invention may contain both colloidal metals and solid metal flakes, preferably made of gold or other noble metal. In further embodiments, the metal particles (e.g., gold) are in the form of flakes, leaves, powder or shavings. Although the terms “flakes,” “particles,” “leaves,” “powder,” and/or “shavings,” are used to describe the metal's appearance and form, these labels are for convenience and do not indicate the specific shape, thickness, and/or other physical properties.

For some transparent or translucent embodiments, the metal flakes will typically be seen to settle to the bottom of the container, but when the composition is shaken, the metal flakes will float throughout the medium and will eventually settle by gravitational forces. It other embodiments, the medium is of such viscosity so as to maintain a more distributed suspension of metal flakes throughout the composition.

In further embodiments, noble metal flakes, such as gold, will be clearly visible in a transparent or essentially transparent medium and will create a sense of luxury and opulence in the user. In still further embodiments, the metal flakes are present in sufficient amounts to provide an aesthetically distinct metal-sprinkled appearance. Yet at the same time, the metal particles will not be noticeable to any great degree when the product is applied in use.

The size of the metal particles can vary widely. In some embodiments, the metal flakes are of a size, shape, texture, thickness, etc. so that when the composition is administered, the particles will disappear or be essentially invisible to the unaided eye. In some embodiments, the size of the metal flakes range from about 1-5 millimeters (mm).

In some embodiments, the colloidal metal and/or metal flakes comprise alloys containing at least two metals. In some embodiments, the alloys include those with about 50% Ag, about 50% Au, about 50% Cu, about 60% Au, about 20% Ag, or about 20% Cu. Furthermore, the metals described in the present invention may be of either crystalline and/or amorphous structure. In some embodiments, the crystalline and amorphous metal particles may be separated.

Preferably, the compositions comprise a colloidal noble metal with antimicrobial, anti-inflammatory, and anti-oxidizing/peroxidizing properties. In preferable embodiments, colloidal gold is present in such amounts so as to have an antimicrobial, antigen, anti-inflammatory, anti-oxidizing/peroxidizing, anti-aging, anti-wrinkling, integument and skin-supporting, and wound-recovering effect on the applied surface. In additional preferable embodiments, gold flakes are visibly dispersed and suspended in the composition. In further preferred embodiments, the composition comprises copper, either in colloidal form or both colloidal and flake form.

Additional Agents

Optionally, in some embodiments, the formulations can include one or more components which can be biologically active or relatively biologically inactive components.

Without wishing to be bound by any theory, it is believed that colloidal metals, such as colloidal gold, bring the additional agents, to be described herein, directly to the extracellular matrix. In some embodiments, colloidal metal is one of many beneficial components in the present invention, wherein some or all of the components are encapsulated by a protective membrane, such as a liposome.

In some embodiments, the composition comprises at least one additional active agent. As defined herein, an “active agent” includes an agent that exhibits antimicrobial activity, antigen activity, antibacterial activity, antiviral activity, antifungal activity, antiparasitic activity, antiprotozoal activity, anti-inflammatory activity, analgesic activity, anti-oxidizing activity, anti-aging activity, anti-wrinkling activity, anti-peroxidizing activity, wound recovery activity, and integument and skin-supporting activity.

The active agent may have multiple functions, such as acting as an anti-inflammatory agent, antioxidant, and an antimicrobial agent. Although described with specific examples, one of skill in the art will recognize that the active as well as inactive agents described herein are illustrative rather than exhaustive.

Antimicrobial

Accordingly, in some embodiments, the antimicrobial agent may be an antigen, antibacterial, antifungal, antiviral, antiparasitic, and an antiprotozoal agent. In some embodiments, the additional antimicrobial agent is selected from triclosan, povidone, iodine, proflavine, honey, hydrogen peroxide, clotrimazole, or sulfur.

In other embodiments, suitable antiviral agents include, for example, virus-inactivating agents and nucleotide or nucleoside analogs, such as tenofovir, acyclovir, tamvir, penciclovir, amantadine, didanosine, foscarnet, ganciclovir, ribavirin, vidarabine, zalcitabine, and zidovudine. Further antiviral agents that may be used include non-nucleoside reverse transcriptase inhibitors, such as UC-781 (thiocarboxanilide), pyridinones, TIBO, nevaripine, delavirdine, calanolide A, capravirine, and efavirenz.

Other antiviral agents that may be used in combination with colloidal metals are those in the category of HIV entry blockers, such as cyanovirin-N, clyclodextrins, carregeenans, sulfated or sulfonated polymers, mandelic acid condensation polymers, monoclonal antibodies, chemokine receptor antagonists, and fusion inhibitors.

Suitable antibacterial agents include antibiotics, such as aminoglycosides, cephalosporins, macrolides, including erythromycins, penicillins, including natural penicillins, penicillinase-resistant penicillins, aminopenicillins, extended spectrum penicillins, sulfonamides, tetracyclines, fluoroquinolones, and metronidazole.

Suitable antifungal agents include amphotericin B, nystatin, griseofulvin, flucytosine, fluconazole, potassium iodide, intraconazole, clortrimazole, miconazole, ketoconazole, and tolnaftate.

Suitable antiprotozoal agents include antimalarial agents, such as chloroquine, primaquine, pyrimethamine, quinine, fansidar, and mefloquine; amebicides, such as dioloxamide, emetine, iodoquinol, metronidazole, paromomycine and quinacrine; pentamidine isethionate, atovaquone, and eflornithine.

Anti-Inflammatory Agents

In addition to antimicrobial agents, some embodiments contain additional anti-inflammatory agents such as anti-inflammatory metals, nonsteroidal anti-inflammatory agents, and/or natural anti-inflammatory substances, for example, hyssop, licorice extract, salicylic acid, bisabolol, fumaric acid, aloe vera, allantoin, and chamomile.

Analgesics

Further embodiments of the compositions of this invention are compositions that include analgesics. The analgesics may comprise amine-containing local anesthetics such as lidocaine and procaine. The analgesics may include, but are not limited to: methyl salicylate, methyl nicotinate, lidocaine, benzyl alcohol, resorcinol, menthol, diphenylhydramine hydrochloride, paracetamol, NSAIDs, benzocaine, butamben picrate, dibucaine, dibucaine hydrochloride, dimethisoquin hydrochloride, dyclonine hydrochloride, lidocaine, lidocaine hydrochloride, pramoxine hydrochloride, tetracaine, tetracaine hydrochloride, alcohols and ketones, camphor, camphorated metacresol, juniper tar, menthol, phenol, phenolate sodium, antihistamines, tripelennamine hydrochloride, hydrocortisone preparations (e.g., hydrocortisone and/or hydrocortisone acetate), allyl isothiocyanate, ammonia solutions, turpentine oil, histamine dihydrochloride, capsaicin, capsicum, and capsicum oleoresin.

Some embodiments of the present invention may comprise a percentage by weight of the compositions listed: benzocaine from about 5-20 percent; butamben picrate from about 1 percent; dibucaine from about 0.25-1 percent; dibucaine hydrochloride from about 0.25-1 percent; dimethisoquin hydrochloride from about 0.3-0.5 percent; dyclonine hydrochloride from about 0.5-1 percent; lidocaine hydrochloride from about 0.5-4 percent; lidocaine from about 0.5-4 percent; pramoxine hydrochloride from about 0.5-1 percent; tetracaine from about 1-2 percent; tetracaine hydrochloride from about 1-2 percent; benzyl alcohol from about 10-33 percent; camphor from about 0.1-3 percent; camphor from about 3-11 percent; camphor combined with phenol with camphor from about 3-10.8 percent; phenol combined with camphor, wherein phenol comprises from about 4.7 percent; camphorated metacresol from about 3-10.8 percent; camphor from about 1-3.6%; metacresol 1 to 3.6 percent; juniper tar from about 1-5 percent; menthol from about 0.1-1 percent; phenol from about 0.5-1.5 percent; resorcinol from about 0.5 to 3 percent; diphenhyldramine hydrochloride from about 1-2 percent; tripelennamine hydrochloride from about 0.5-2 percent; hydrocortisone from about 0.25-0.5 percent; hydrocortisone acetate from about 0.25-0.5 percent; allyl isothiocyanate from about 0.5-5 percent; ammonia solution from about 1-2.5 percent ammonia; methyl salicylate from about 10-60 percent; turpentine oil from about 6-50 percent; menthol from about 1.25-16 percent; histamine dihydrochloride from about 0.025-0.10 percent; methyl nicotinate from about 0.25-1 percent; capsaicin from about 0.025-0.25 percent; capsicum containing from about 0.025-0.25 percent capsaicin; and/or capsicum oleoresin containing from about 0.025-0.25 percent capsaicin.

Antioxidants

Some embodiments may also include antioxidants such as superoxide dismutase, beta glucan, idebenone, curcumin, carnosin, polyphenolics, catalase, glutathione peroxidase, oligomeric proanthocyanidins, bioflavonoids, leuco anthocyanin, anthocyanidin, alpha-lipoic acid, coenzyme Q10, selenium, ferulic acid, caffeic acid, and vitamin C.

Antigen

Some embodiments may also include antigens such as, for example, proteins (e.g. antibodies), polypeptides, carbohydrates, polymers, polysaccharides, enzymes, and nucleic acids.

### Sebum-Reducing Agent

Sebum consists of a mixture of squalane wax esters, cholesterol esters, and triglycerides. An abnormally high rate of sebum supports the growth and proliferation of the propionibacterium that causes acne. Further embodiments of the present invention may include sebum reducers such as azelaic acid, revivogen, and/or MK-386 (4,7-[beta]-dimethyl-4-aza-5a-cholestan-3-one).

Hormones and Growth Factors

Further embodiments may also comprise hormones and growth factors such as kinetin, chemokines, epidermal growth factor EGF, fibroblast growth factor FGF, platelet-derived growth factor (PDGF), growth hormone, melatonin, pregnenolone, progesterone, testosterone, interleukin and transforming growth factor TGF.

Enzymes

Additional embodiments can include enzymes such as proteases (e.g., metalloproteinases) kinases, tyrosine and serine kinases, lysozyme, procollagenase, etc.

Peptides

The present invention also provides for compositions comprising peptides. The peptides of the invention include those that can assist wound recovery and healing, treat and prevent skin and integument conditions, and mitigate the effects of skin-aging. Individual peptides, peptide variants, peptide derivatives and mixtures thereof (e.g. those with different sequences) can be combined in a formulation to promote wound healing and to prevent or treat skin and integument problems. These peptides include, for example, palmitoyl pentapeptide, ubiquitin, oligopeptide, neuropeptide Y, pentapeptide, hexapeptide, argirilene (hexapeptide-3), acetyl hexapeptide-3, palmitoyl pentapeptide 3, epidermal growth factor (EGF), copper peptides, and fibroblast growth factor (FGF).

Proteins and Amino Acids

In certain embodiments, the present invention further provides for compositions comprising proteins, such as fibronectin, or amino acids, including alanine, arginine, asparagine, aspartic acid, cysteine, cystine, glutamine, glutamic acid, glycine, histidine, isoleucine, leucine, lysine, methionine, phenylalanine, proline, serine, threonine, tryptophan, tyrosine, or valine.

### Lipids

In some further embodiments, the present invention further comprises lipids. These can include naturally occurring and synthetic lipids. Lipids are well known in the art, and include for example, nautral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. In some embodiments, the composition further comprises glycerides, phospholipids, lecithin, and/or phosphatidylcholine.

Retinoids

Some embodiments may include retinoids such as retinol and retinyl esters. For example, Retinol (vitamin A) is an endogenous compound which occurs naturally in the human body and is essential for normal epithelial cell differentiation. Natural and synthetic vitamin A derivatives have been used extensively in the treatment and prevention of a variety of skin disorders and have been used as anti-aging, anti-wrinkling, skin repair, and/or renewal agents. Retinoic acid has been employed to treat a variety of skin conditions, e.g., acne, wrinkles, psoriasis, age spots, and discoloration. Other retinoids that may be included are, for example, tretinoin, retinol, rose hips, and 9-cis retinoic acid.

Vitamins

The present invention also provides for compositions containing vitamins. These include, for example, vitamin A, B1, B2, B3, B5, B6, B7, B9, B 12, C, Ester-C, D, E, F, and K. Other embodiments may comprise vitamin containing compounds such as Rose Hips.

Wound Recovery Agents

In some embodiments, the compositions further comprise wound recovery agents. The term “wound recovery” refers generally to activity promoting the healing and repair of injured or dramatized tissue. The agent may work to promote healing, for example, by disinfecting, reducing inflammation, and pain-relief. Suitable wound recovery agents include allantoin, geranium extract, azelaic acid, curcumin, fumaric acid, gamma linolenic acid, farsenol, and squalene.

Botanical Extracts

Compositions of the present invention may also include botanical extracts, for example, aloe, witch hazel, chamomile, licorice extract, and hydrogenated soy oil.

MMP Inhibitors

Matrix metal loproteinases (MMPs) refer to any protease of the family of MMPs which are involved in the degradation of connective tissues, such as collagen, elastin, fibronectin, laminin, and other components of the extracellular matrix. Examples of MMPs include MMP-2 (secreted by fibroblasts and a wide variety of other cell types) and MMP-9 (released by mononuclear phagocytes, neutrophils, corneal epithelial cells, tumor cells, cytotrophoblasts and keratinocytes). Conditions characterized by undesirable MMP activity include ulcers, skin disorders, skin aging, wounds, cancer including cell proliferation, and collagenase induced disease.

In some embodiments, MMP inhibitors include alkanolamine MMP inhibitors comprising alkanolamines selected, for example, from ethylaminoethanol, methylaminoethanol, dimethylaminoethanol (DMAE), isopropanolamine, triethanolamine, isopropanoldimethylamine, ethylethanolamine, 2-butanolamine, choline, serine, and mixtures thereof, are known in the art to promote skin repair and renewal. In other embodiments, the MMP inhibitor is selected from minimal-domain MMPs, simple hemopexin domain-containing MMPs, gelatin-binding MMPs, furin-activated MMPs, and vitronectin-like insert MMPs, type I transmembrane MMPs, glycosyl-phosphatidyl inosital (GPI)-linked MMPs, and type II transmembrane MMPs. In further embodiments, the MMP inhibitor is a DMAE MMP inhibitor.

Ceramides

Ceramides belong to the lipidic constituents of the stratum corneum. It is believed that ceramides may have beneficial effects on skin and integument tissues. See U.S. Pat. No. 6,962,697. In some embodiments, the present invention further comprises ceramides.

Sunscreen Components

Sunscreens generally absorb ultraviolet radiation. Some embodiments of the present invention provide for sunscreen containing compositions. These embodiments can include, for example, oxybenzone(2-hydroxy-4-methoxybenzophenone), doxybenzone(2,2′)-dihydroxy-4-methoxybenzophenone), aminobenzoic acid, cinoxate(2-ethoxyethyl-p-methoxycinnamate), diethanolamine-p-methoxycinnamate, digalloyltrioleate ethyl-4-bis(hydroxypropyl)aminobenzoate, 2-ethylhexyl salicylate, glyceryl aminobenzoate, homosalate(3,3,5-trimethylcyclohexyl salicylate, triethanolamine salicylate, 2-phenylbenzimidazole-5-sulfonic acid, sulisobenzone(2-hydroxy-4-methoxybenzophenone-5-sulfonic acid), Padimate A (amyl p-dimethylaminobenzoate), Padimate O (octyl dimethyl p-aminobenzoate), 4-t-butyl-4′-methoxydibenzoylmethane, and the combination of 2-hydroxy-1,4-naphthoquinone with dihydroxyacetone and methyl anthranilate.

Sunblock Components

Sunblocks work by physically blocking radiation from reaching the skin. In some embodiments, the present invention provides for sunblock containing compositions to protect the skin for ultraviolet radiation. Suitable sunblocking agents include, but are not limited to titanium dioxide, aluminum oxide, magnesium dioxide, and zinc oxide.

Polysaccharides

In additional embodiments, the present invention provides for polysaccharide containing compositions. Suitable polysaccharides include beta glucans such as, for example, dextran. Glucan is a naturally occurring component of cell walls in some bacterial organisms, yeast, grain endosperm, and fungus. Glucans, specifically beta glucans, have been found to have many beneficial properties when administered. For example, studies have shown that beta glucan can activate immunological responses from patients. (Pelizon et al., Physiol, Res. 2005, 54, 557-564). Although the specific mechanism by which beta glucans provide benefits is still unclear, it is believed that the presence of glucans and beta glucans protects and stimulates gene regulation and function. For example, it is believed that the presence of glucans and/or beta glucans stimulates collagen synthesis and wound recovery.

Integument and Skin-Supporting Components

In further embodiments, the present invention further comprises one or more integument and skin-supporting components. In some embodiments, the integument/skin supporting component may be selected from the group consisting of vitamin K, beta glucan, borage oil, flax seed, cod liver oil, black currant, alpha and beta hydroxy acids, grape seed, pycnogenol, rose hips, sunscreens, tea tree oil, acetyl glucosamine algae, collagen, elastin, copper, dead sea minerals, glycerin, hormone creams, human growth factor, kinetin, lanolin, mineral oil, olive oil, oxygen, perfluorodecalin, soy lecithin phospholipids, hydrogen peroxide, triclosan, salicylic acid, papain, aloe vera, lavender oil, geranium oil, chamomile, calendula officinalis, squalane, magnesium oxide crystals, macademia nut oil, galactoarabinan, magnesium aluminum silicate, sweet almond oil, sesame oil, palmitoyl-pentapeptide-3, peptides, benzoic acid, butylene glycol, carbomer, phyllanthus emblica fruit extract, urea, centella asiatica extract, echinacea angustifoila (coneflower) extract, hydrolyzed wheat protein, propylene glycol, bearberry extract, licorice, carnosine, caffeine, cocoa butter, kukui nut, shea butter, mugwort extract (artemesia vulgaris), mango butter, plantago lanceolate leaf extract, xanthan gum, sodium lauryl sulfate, glycolic acid, lactic acid, malic acid, citric acid, aartaric acid, all-trans retinoic acid, allantoin, aloe barbadensis, aminobutyric acid, arbutin, arginine amino acid, azelaic acid, caffeic acid, ferulic acid, carnosine, retin-A, ceramides, copper gluconate, superoxide eismutase, curcumin, cystosine, dehydroepiandrosterone, DHEA, dinitrochlorobenzene, dipotassium glycyrrhizinate, hydantoin, DMSO, elastin, Ester-C, bromelian, erythropoietin (EPO), evening primrose oil, farnesol, fumaric acid, GABA (gamma aminobutyric acid/gamma amino-butyric acid), gamma linolenic acid, geranium extract, glutathione, glycyrrhiza glabra glycyrrhetic acid, hesperidin, hyaluronic acid, N6-Furfuryladenine Hormone, kojic acid, Tissue Inhibitors Of Metal loproteinases Matrixyl (TIMPS), vitamin E, vitamin C, flavonoids, palmitoyl panax ginseng root extract, pantothenic acid, pycnogenol scavenger, phosphatidylcholine, resveratrol, retinol, silicone, soy extracts, squalene, sulfur, saw palmetto, tocotrienols, ubiquinone, coenzyme Q10, botox, botulinum toxin, IGF, calendula officinalis, immortelle, green tea extract, white tea, black tea, glucosamine, algae, yeast, magnesium, magnesium aspartate, glycerin, progesterone, estrogen, lavender, cucumber, DNA, panthenol, zinc gluconate, camellia oleifera leaf extract, and pomegranate.

Anti-Aging and Anti-Wrinkling Agents

Though not separately described in this section, many of the aforementioned substances have anti-aging and anti-wrinkling effects. The terms “anti-wrinkling” and “anti-aging” agents refer generally to substances and materials that provide integument and skin-supporting benefits by promoting and/or maintaining the natural process of desquamation; preventing skin damage by protection against photoaging and sun damage; promoting skin repair and renewal; and/or hydrating the skin. In some embodiments, the anti-aging and/or anti-wrinkling agents may prevent damage contributing to skin's deterioration or may promote repair and renewal such that the skin may, for example, attain a more youthful and healthy appearance.

Other Additional Agents

In some embodiments, other suitable additional agents may be included in the compositions. These include color agents, such as glitter, dyes, colorants, and/or pigments; flavoring or scenting agents such as peppermint oil; temperature agents to generate warmth, coolness, or otherwise affect the perception of temperature; essential oils; and/or massage oils.

These massage and/or essential oils can include seed oils, oils of mandarin, (Citrus reticulata var. mandarin), sage (Salvia officinalis), geranium rose (Pela graveolens xasperium), palmarosa (Cynbopogon martini), nutmeg (Myristica frangrans), rosewood (Aniba roseaodora), cedarwood (Junipems virginiana), patchouli (Pogostemon cablin), cardamom (Elettaria cardamomum), vetiver (Vetivertia zizanioides), orange (Citru sinensis L. osbeck), sandalwood (Santalum album), clary sage (Salvia sciarea), rose (Rosa centifolia), jasmine, (Jasminum grandiflorum), yarrow (Achillea millefolium), tanacetum (Tanacetum annuum), ylang ylang (Cananga odorata), rosemary (Rosmarinus officinalis), birch (Betulalenta), grapefruit (Citrus paradisi), cypress (Cupressus sempervirens), peppermint (Mentha piperita), bay laurel (Laurus nobilis), black pepper (Piper nigrum), ginger root (Zingiber officinale), juniper berry (Juniperus communis), lemongrass (Cymbopogon flexuosus), and wintergreen (Gaultheria procumbens).

Pharmaceutically Acceptable and/or Suitable Carriers

As used herein, a “pharmaceutically acceptable,” “acceptable,” and/or “suitable” carrier includes any and/or all solvents, dispersion media, coatings, isotonic, and/or absorption delaying agents and/or the like. The phrases “pharmaceutically,” “pharmacologically,” “suitable,” and/or “acceptable” refer to materials, substances, or compositions that do not produce an adverse, allergic or other untoward reaction when administered to a subject. The selection and use of such materials may be readily determined by one of skill in the art.

Carriers can optionally include one or more components which can be biologically active or inactive. Examples of such optional inactive components include base components (e.g., water, propylene glycol, glycerol, polyethylene glycols, silicones, and/or an oil, such as liquid paraffin, vegetable oil, peanut oil, castor oil, and cocoa butter), surfactants, thickening agents (e.g., aluminum stearate and hydrogen lanolin), gelling agents, stabilizing agents, emulsifying agents, dispersing agents, suspending agents, humectants, emollients, acidic or alkaline substances, buffering agents, anti-crystalline agents, lubricating agents, coloring agents, perfumes, excipients (e.g., starch, tragacanth, and cellulose derivatives), foaming agents, diluents, fillers, binding agents, and preservatives (e.g., methyl paraben, propyl paraben, methylchloroisothiazolinone, and methylisothiazolinone). Suitable carriers also include water-based, silicone-based, petroleum-based, natural-oil based, and massage formulations.

In some embodiments, the carrier may comprise capsules suitable for depositing actives, fragrances, color, glitter etc. onto the skin and integument. Capsules suitable for deposition include, for example, capsules made from mannitol, lactose cellulose, and hydroypropylmethylcellulose. Suitable capsules are, for example, marketed using the Unispheres process of Inducehm, Dubendorf, Switzerland.

The present invention also provides for skin-penetrating or skin-permeating carriers. These include, for example, DMSO, liposomes, lipophilic solvents, lecithin, transcutol, nanospheres, nanoshells, and rovisomes.

Delivery Structures

In other embodiments of the present invention, the components of the compositions are encapsulated in a protective membrane structure. The protective membrane structure can be, for example, a hollow and solid lipid structure, nanoshell, nanosphere, cerasome, rovisome, or nano-structure.

By way of example, in particular embodiments, the encapsulating protective structure is a liposome. A liposome is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that can comprise an aqueous composition.

In some embodiments, some or all of the components of the present invention may be encapsulated in the aqueous interior of a liposome, interspersed within the lipid bilayer of a liposome, attached to a liposome via a linking molecule that is associated with the liposome, entrapped in a liposome, complexed with a liposome, etc. The size of a liposome varies depending on the method of synthesis. Liposomes in the present invention can be a variety of sizes. In preparing such liposomes, any protocol as would be known to one of ordinary skill in the art may be used.

In the context of the present invention, the phrase “Protegenetic liposome” or “Protegenetic liposome complex” generally refers to some embodiments of the present invention, wherein a protective liposomal membrane encapsulates some or all of the components in the skin and integument compositions described.

In further embodiments, more than one protective membrane may be used. For example, in some embodiments, the composition of the present invention comprises a first protective membrane encapsulating one or more components of the composition and a second protective membrane encapsulating the first protective membrane and additional components not encapsulated by the first protective membrane. In another embodiment, the composition comprises a first protective membrane encapsulating one or more components of the composition and at least one additional protective membrane encapsulating one or more components of the composition. Regardless of whether a single or multiple protective membranes are used, each individual membrane can be designed to encapsulate a portion of the components of the composition or all components of the composition.

Without wishing to be bound by any theory, it is believed that the Protegenetic liposome complex protects genes and gene function by providing supplemental additional agents such as antigens, proteins, and antioxidants to prevent and repair gene damage. For example, it is believed that the antioxidizing and anti-peroxidizing properties of the components encapsulated by the Protegenetic liposome neutralize free radicals that may damage genes and hamper the production of regenerative hormones and enzymes essential for collagen and fibrin production.

Formulations

The present invention also provides for formulations suitable for various routes of administering the described composition to an animal. These include, for example, topical, oral, transdermal, mucosal, enteral, parenteral, and sublingual formulations. The formulations may be in the form of, for example, a lotion, ointment, paste, foam, emulsion, cream, serum, aerosol, spray, roll-on formulation, masque, microdermabrasion formulation, cleanser, moisturizer, pill, tablet, caplet, capsule, gel-cap, and transdermal patch.

In certain embodiments, the described composition comprises carrier components useful for topical application to skin, integument, and mucosal membrane. This composition can be, for example, a cream, ointment, lotion, cosmetic, soap, wash, shampoo, conditioner, rinse, hair tonic, hair spray, hair dye, hair care treatment, scalp treatment, pain relief spray, facial spray, roll-on formulation, masque, cleanser, moisturizer, and composition-containing pad, patch, strip, or bandage etc. The topical formulation can include other ingredients such as humectants that reduce the rate at which the cream or lotion dries out.

Oral formulations can include the use of compositions suitable for administration to the mouth, gums, or teeth. Such formulations can include oral hygiene products, for example, tooth pastes, mouthwashes, chewing gum, tooth-whitening formulation, dental balm etc.

Transdermal formulations can include the use of skin-permeating components, for example, DMSO, liposomes, lipophilic solvents, lecithin, transcutol, nanospheres, nanoshells, and rovisomes.

Additional formulations are suitable for other modes of administration to mucosal membranes such as the nasal lining (e.g., nasal spray), vaginal, buccal, and/or rectal surfaces. For vaginal or rectal surfaces, a pessary and/or suppository may also be used. Suppositories are solid dosage forms of various weights and/or shapes, usually medicated, for insertion into the rectum, vagina and/or the urethra. After insertion, suppositories soften, melt and/or dissolve in the cavity fluids. In general, for suppositories, traditional binders and/or carriers may include, for example, polyalkylene glycols and/or triglycerides.

Enteral formulations include such normally employed excipients as, for example, mannitol, lactose, starch, magnesium stearate, sodium saccharine, cellulose, magnesium carbonate or the like. In certain embodiments, oral compositions will comprise an inert dilutent or edible carrier, and/or they may be enclosed in hard or soft shell gelatin capsule, and/or they may be compressed into tablets, or they may be incorporated directly with the food of the diet.

Tablets, troches, pills, capsules and/or the like may also contain the following: a binder, such as gum tragacanth, acacia, cornstarch, or gelatin; excipients, such as dicalcium phosphate; a disintegrating agent, such as corn starch, potato starch, alginic acid or the like; a lubricant, such as magnesium stearate; a sweetening agent, such as sucrose, lactose or saccharin may be added; or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. In some embodiments, a syrup or elixir may contain sucrose as a sweetening agent and a dye for color.

Additionally, the present invention provides for parenteral administration in, for example, an aqueous solution. Such a solution should be suitably buffered if necessary and/or the liquid diluent first rendered isotonic with sufficient saline and/or glucose. In some embodiments, the aqueous solutions may be suitable for intravenous, intramuscular, subcutaneous or intraperitoneal administration. In this connection, sterile aqueous media which can be employed will be known to those of skill in the art in light of the present disclosure. As with all the formulations, some variation in dosage will necessarily occur depending on the condition of the subject being treated.

In further embodiments, the present invention provides for a composition for sublingual administration. The sublingual formulation may comprise conventional pharmaceutical additives and excipients used in the art for sublingual preparations.

Additional Formulations

In addition to the embodiments disclosed, the present invention also provides for additional formulations as described herein.

In some embodiments, the present invention provides for a composition comprising colloidal gold, EGF, beta glucan, Copper/PCA, superoxide dismutase, ceramide II, transcutol, hyaluronic acid, squalene, and rejuvenox. Preferably, some or all of the components are encapsulated by a protective membrane such as a liposome or nanosphere.

Topical Cream

In one embodiment, the present invention provides for a topical cream composition comprising Water, Octyldodecanol. Caprylic/Capric Triglyceride, Dimethicone, Ethyl Macadamiate, Cetyl Esters, Tribehenin, Tribehenin PEG-20 Esters, Dimethyl Lauramine Oleate, Butyrospermum Parkii (Shea Butter), Palmitoyl Oligopeptide (Biopeptide CL™), Polysorbate 80, Cyclomethicone, Cetearyl Octanoate, Propylene Glycol, Soluble Collagen, Algae Extract, Sodium Hyaluronate, Colloidal Gold, Immortelle, Beta Glucan, Colloidal Trace Minerals, Gold Unipheres, Dimethicone PEG-8 Meadowfoamate, Sodium PCA, Hydroxyethyl Urea, Retinyl Palmitate (Vitamin A Palmitate), Tocopheryl Acetate (Vitamin E Acetate), Panthenol (Vitamin B5), Aloe Barbadensis (Aloe Vera) Leaf Juice, Centella Asiatica (Gotu Kola) Extract, Calendula Officinalis (Marigold) Flower Extract, Glycyrrhiza Glabra (Licorice) Root Extract, Camellia Sinensis (Green Tea) Leaf Extract, Juglans Regia (Walnut) Seed Extract, Rosa Mosqueta (Rose Hip) Oil, Glucosamine HCl, Yeast Extract, Urea, Fragrance, Glyceryl Stearate, Beeswax, Tocopherols, Bisabolol, Xanthan Gum, Ammonium Acryloyldimethyltaurate/VP Copolymer, Sodium Acrylate/Sodium Acryloydimethyl Taurate Copolymer, Ethylhexylglycerin, and Phenoxyethanol.

Masque

In another embodiment, the present invention provides for a masque composition comprising at least one component selected from the group consisting of water, sodium magnesium silicate, glycerin, mica, titanium dioxide, polyquaternium-7, hydroxyethyl urea, polyacrylamide, C 13-14 isoparaffin, laureth-7, immortelle essential oil, colloidal gold, dipalmitoyl hydroxyproline, tetrahexyldecyl ascorbate, C 12-15 alkyl benzoate, tribehenin, ceramide II, PEG-40 rapeseed sterol, palmitoyl oligopeptide, bismuth oxychloride, magnesium sulfate, calcium carbonate, zinc oxide, propylene glycol, methylparaben, propylparaben, potassium sorbate, and fragrance. Preferably, in some non-limiting embodiments, the masque composition comprises components of a percentage by weight listed below:

| | |
|---|---|
| Water | from about QS to 100.00% |
| Sodium Magnesium Silicate | from about 2.0-10.00% |
| Glycerin | from about 1.0-10.00% |
| Mica | from about 0.05-3.000% |
| Titanium Dioxide | from about 0.05-5.000% |
| Polyquaternium-7 | from about 0.05-1.000% |
| Hydroxyethyl Urea | from about 0.05-5.000% |
| Polyacrylamide | from about 0.05-0.500% |
| C 13-14 Isoparaffin | from about 0.10-1.000% |
| Laureth-7 | from about 0.05-1.000% |
| Helichrysum Angustifolium | from about 0.001-1.00% |
| (Immoretelle) Extract | |
| Colloidal Gold | from about 0.001-1.00% |
| Dipalmitoyl Hydroxyproline | from about 0.001-1.00% |
| Tetrahexyldecyl Ascorbate | from about 0.001-1.00% |
| C 12-15 Alkyl Benzoate | from about 0.001-1.00% |
| Tribehenin | from about 0.001-0.50% |
| Ceramide II | from about 0.001-0.50% |
| PEG-40 Rapeseed Sterol | from about 0.001-0.50% |
| Palmitoyl Oligopeptide | from about 0.0001-0.5% |
| Bismuth Oxychloride | from about 0.05-1.000% |
| Magnesium Sulfate | from about 0.0001-1.0% |
| Calcium Carbonate | from about 0.0001-1.0% |
| Zinc Oxide | from about 0.0001-1.0% |
| Propylene Glycol | from about 0.01-2.000% |
| Methylparaben | from about 0.05-0.300% |
| Propylparaben | from about 0.05-0.300% |
| Potassium Sorbate | from about 0.01-0.300% |
| Fragrance | from about 0.01-0.500% |
| Yellow 5 | To match std. |
| Red 40 | To match std. |
| Blue 1 | To match std. |

In further non-limiting embodiments, the masque composition comprises components of a percentage by weight listed below:

| | |
|---|---|
| Water | A |
| Sodium Magnesium Silicate | C |
| Glycerin | C |
| Colloidal Gold (24K) | D |
| Mica (Gold) | E |
| Titanium Dioxide | E |
| Polyquaternium-7 | E |
| Hydroxyethyl Urea | E |
| Polyacrylamide | E |
| C13-14 Isoparaff[iota]n | E |
| Laureth-7 E Helichrysum Angustifolium (Immortelle) Extract | E |
| Bismuth Oxychloride | E |
| Ricinus Communis (Castor) Seed Oil | E |
| Phenoxyethanol | E |
| Ethylhexylglycerin | E |
| Dipalmitoyl Hydroxyproline | F |
| Tetrahexyldecyl Ascorbate | F |
| Fragrance | F |
| Magnesium Sulfate | F |
| Calcium Carbonate | F |
| Zinc Oxide | F |

Composition Legend

A: from about 30% to about 100%

B: from about 10% to less than about 30%

C: from about 3% to less than about 10%

D: from about 1% to less than about 3%

E: from about 0.1% to less than about 1%

F: less than about 0.1%

In additional embodiments, the masque composition further comprises Protegenetic liposomes. In some embodiments, the Protegenetic liposomes comprise from about 0.01-1.0 percent by weight of the composition.

Serums

In another embodiment, the present invention provides for a serum composition comprising at least one component selected from the group consisting of cyclopentasiloxane, dimethicone, vinyl dimethicone copolyol crosspolymer, immortelle essential oil, colloidal gold, gold leaf, dipalmitoyl hydroxyproline, and tetrahexyldecyl ascorbate. Preferably, in some non-limiting embodiments, the serum composition comprises components of a percentage by weight listed below:

| | |
|---|---|
| Cyclopentasiloxane | from about QS to 100.00% |
| Dimethicone/Vinyl Dimethicone | from about 0.01-50.0000% |
| Crosspolymer | |
| Helichrysum Angustifolium | from about 0.0001-1.000% |
| (Immortelle) Extract | |
| Colloidal Gold | from about 0.0001-2.000% |
| Gold leaf | from about 0.0001-1.000% |
| Dipalmitoyl Hydroxyproline | from about 0.0010-1.000% |
| Tetrahexyldecyl Ascorbate | from about 0.0010-1.000% |

In further non-limiting embodiments, the serum composition comprises components of a percentage by weight listed below:

| | |
|---|---|
| Cyclopentasiloxane | A |
| Dimethicone/Vinyl Dimethicone Crosspolymer | C |
| Helichrysum Angustifolium (Immortelle) Extract | F |
| Gold Leaf (CI 77480) | F |
| Dipalmitoyl Hydroxyproline | F |
| Tetrahexyldecy] Ascorbate | F |
| Colloidal Gold (24K) | F |

Composition Legend

A: from about 30 to about 100%

B: from about 10% to less than about 30%

C: from about 3% to less than about 10%

D: from about 1% to less than about 3%

E: from about 0.1% to less than about 1%

F: less than about 0.1%

In additional embodiments, the serum composition further comprises Protegenetic liposomes. In some embodiments, the Protegenetic liposomes comprise from about 0.01-1.0 percent by weight of the composition.

Anti-Wrinkle

In an additional embodiment, the present invention provides for an anti-wrinkle composition comprising at least one component selected from the group consisting of water, sodium silicate, magnesium aluminum silicate, prunus amygdalus dulcis extract, cellulose gum, titanium dioxide, aloe barbadensis juice, colloidal gold, chamomilla recutita flower extract, methylparaben, propylparaben, diazolidinyl urea, and iron oxides (CI 77489, CI 77491, CI 77492, CI 77499). Preferably, in some non-limiting embodiments, the anti-wrinkle composition comprises components of a percentage by weight listed below:

| | |
|---|---|
| water | from about QS to 100.00% |
| sodium silicate | from about 5.0-10.0% |
| magnesium aluminum silicate | from about 2.0-4.0% |
| prunus amygdalus eulcis extract | from about 2.0-4.0% |
| cellulose gum | from about 1.0-2.0% |
| titanium dioxide | from about 0.5-2.0% |
| aloe barbadensis juice | from about 0.1-0.5% |
| colloidal gold | from about 0.01-2.0% |
| chamomilla recutita flower extract | from about 0.01-0.1% |
| methylparaben | from about 0.1-0.3% |
| propylparaben | from about 0.1-0.2% |
| diazolidinyl urea | from about 0.2-0.3% |
| iron oxides | up to about 1.0% |

In further non-limiting embodiments, the anti-wrinkle composition comprises components of a percentage by weight listed below:

| | |
|---|---|
| Water | A |
| Sodium Silicate | C |
| Colloidal Gold (24K) | D |
| Magnesium Aluminum Silicate | D |
| Prunus Amygdalus Dulcis Extract | D |
| Cellulose Gum | D |
| Titanium Dioxide | E |
| Aloe Barbadensis Juice | E |
| Chamomilla Recutita Flower Extract | E |
| Phenoxyethanol | E |
| Iron Oxides (CI 77489, CI 77491, CI 77492, CI 77499) | E |
| Dipalmitoyl Hydroxyproline | F |
| Helichrysum Angustifolium (Immortelle) Extract | F |
| Ethylhexylglycerin | F |

Composition Legend

A: from about 30% to about 100%

B: from about 10% to less than about 30%

C: from about 3% to less than about 10%

D: from about 1% to less than about 3%

E: from about 0.1% to less than about 1%

F: less than about 0.1%

In additional embodiments, the anti-wrinkle composition further comprises Protegenetic liposomes. In some embodiments, the Protegenetic liposomes comprise from about 0.01-1.0 percent by weight of the composition.

Microdermabrasion

In a further embodiment, the present invention provides for a microdermabrasion composition comprising at least one component selected from the group PEG-8, sodium bicarbonate, ethoxydiglycol, magnesium oxide crystals, glycerin, Oleth-20, trihydroxystearin, glyceryl stearate and PEG-100 stearate, Protegenetic liposome, tocopheryl acetate (Vitamin E), retinyl palmitate (Vitamin A), ascorbyl palmitate, tetrahexadecyl ascorbate, immortelle, stearyl glycyrrizinate, annatto, propylparaben, or fragrance. Preferably, in some non-limiting embodiments, the anti-wrinkle composition comprises components of a percentage by weight listed below:

| | |
|---|---|
| PEG-8 | from about 15.0-25.0% |
| Sodium Bicarbonate | from about 15.0-20.0% |
| Ethoxydiglycol | from about 15.0-20.0% |
| Magnesium Oxide Crystals | from about 15.0-25.0% |
| Glycerin | from about 5.0-10.0% |
| Oleth-20 | from about 5.0-10.0% |
| Trihydroxystearin | from about 5.0-10.0% |
| Glyceryl Stearate (and) PEG-100 Stearate | from about 3.0-6.0% |
| Protegenetic Liposome | from about 0.5-2.0% |
| Tocopheryl Acetate (Vitamin E) | from about 0.01-0.05% |
| Retinyl Palmitate (Vitamin A) | from about 0.01-0.05% |
| Ascorbyl Palmitate | from about 0.01-0.05% |
| Tetrahexadecyl Ascorbate | from about 0.01-0.05% |
| Immortelle | from about 0.01-0.05% |
| Stearyl Glycyrrizinate | from about 0.01-0.05% |
| Annato | from about 0.01-0.05% |
| Propylparaben | from about 0.1-0.2% |
| Fragrance | from about 0.1-0.7% |

Cleanser

Additionally, the present invention provides for a cleansing composition comprising at least one component selected from the group consisting of water, ammonium lauryl sulfate, acrylates copolymer, cocamidopropyl betaine, glycerin, disodium lauroamphodiacetate, sodium laureth sulfate, glycol stearate, Protegenetic liposomes, beta glucan, colloidal gold, bromelain, papain, panthenol, salicylic acid, hydrolyzed mucopolysaccharides, aloe barbadensis (aloe vera) leaf extract, sodium PCA, Unispheres, beta carotene, helichrysum (Immortelle) oil, disodium EDTA, triethanolamine, methylchloroisothiazolinone and methylisothiazolinone, and fragrance. Preferably, in some non-limiting embodiments, the cleanser composition comprises components of a percentage by weight listed below:

| | |
|---|---|
| Water | from about 12.60-63.76% |
| Ammonium Lauryl Sulfate | from about 20.0-35.0% |
| Acrylates Copolymer | from about 5.0-10.0% |
| Cocamidopropyl Betaine | from about 5.0-10.0% |
| Glycerin | from about 1.0-4.0% |
| Disodium Lauroamphodiacetate | from about 1.0-4.0% |
| Sodium Laureth Sulfate | from about 1.0-4.0% |
| Glycol Stearate | from about 1.0-4.0% |
| Protegenetic Liposome | from about 1.0-4.0% |
| Beta Glucan | from about 0.1-1.0% |
| Colloidal Gold | from about 0.1-1.0% |
| Bromelain | from about 0.1-1.0% |
| Papain | from about 0.1-1.0% |
| Panthenol | from about 0.1-1.0% |
| Salicylic Acid | from about 0.1-1.0% |
| Hydrolyzed Mucopolysaccharides | from about 0.1-1.0% |
| Aloe Barbadensis (Aloe Vera) Leaf Extract | from about 0.1-1.0% |
| Sodium PCA | from about 0.1-1.0% |
| Unispheres | from about 0.1-1.0% |
| Beta Carotene | from about 0.1-1.0% |
| Immortelle | from about 0.01-.1% |
| Disodium EDTA | from about 0.01-0.1% |
| Triethanolamine | from about 0.1-1.0% |
| Methylchloroisothiazolinone and | from about 0.01-0.1% |
| Methylisothiazolinone | |
| Fragrance | from about 0.01-0.1% |

Moisturizer

In a further embodiment, the present invention provides for a moisturizing composition comprising at least one component selected from the group consisting of water, cyclomethicone, ethylhexyl isononanoate, acetyl hexapeptide-3, sodium PCA, PEG-100 stearate, glyceryl stearate, stearic acid, dimethicone, butyrospermum parkii (Shea Butter), Protegenetic liposome, beta glucan, colloidal minerals, colloidal gold, Unispheres, helichrysum (Immortelle) oil, dimethyl MEA (DMAE), sodium hyaluronate, superoxide dismutase, ascorbyl palmitate, ceramide II, bisabolol, squalane, retinyl palmitate, steareth-2, annatto, magnesium aluminum silicate, carbomer, triethanolamine, methylparaben, DMDM hydantoin, iodopropynyl butylcarbamate, and fragrance. Preferably, in some non-limiting embodiments, the moisturizer composition comprises components of a percentage by weight listed below:

| | |
|---|---|
| Water | from about 50.0-70.0% |
| Cyclomethicone | from about 7.0-12.0% |
| Ethylhexyl Isononanoate | from about 5.0-10.0% |
| Acetyl Hexapeptide-3 | from about 2.0-7.0% |
| Sodium PCA | from about 2.0-7.0% |
| PEG- 100 Stearate | from about 1.0-4.0% |
| Glyceryl Stearate | from about 1.0-4.0% |
| Stearic Acid | from about 1.0-4.0% |
| Dimethicone | from about 1.0-3.0% |
| Butyrospermum Parkii (Shea Butter) | from about 1.0-3.0% |
| Protegenetic Liposome | from about 0.5-2.5% |
| Beta Glucan | from about 0.5-2.5% |
| Colloidal Minerals | from about 0.1-0.5% |
| Colloidal Gold | from about 0.001-50.0% |
| (Unispheres) | from about 0.5-1.0% |
| Immoretelle | from about 0.01-0.05% |
| Dimethyl MEA (DMAE) | from about 0.01-0.05% |
| Sodium Hyaluronate | from about 0.1-0.5% |
| Superoxide Dismutase | from about 0.01-0.05% |
| Ascorbyl Palmitate | from about 0.01-0.05% |
| Ceramide II | from about 0.01-0.05% |
| Bisabolol | from about 0.01-0.05% |
| Squalane | from about 0.5-1.0% |
| Retinyl Palmitate | from about 0.1-0.5% |
| Steareth-2 | from about 0.5-1.0% |
| Annatto | from about 0.01-0.05% |
| Magnesium Aluminum Silicate | from about 0.01-0.05% |
| Carbomer | from about 0.1-0.5% |
| Triethanolamine | from about 0.1-0.5% |
| Methylparaben | from about 0.1-0.5% |
| DMDM Hydantoin | from about 0.1-0.5% |
| Iodopropynyl Butylcarbamate | from about 0.01-0.05% |
| Fragrance | from about 0.1-0.5% |

Pain-Relieving

In an additional embodiment, the present invention provides for a pain-relieving composition comprising at least one component selected from the group consisting of methyl salicylate, menthol, camphor, methyl nicotinate, acrylates/C 10-30 alkyl acrylate crosspolymer, arginine, arnica Montana extract, ascorbyl palmitate, butylene glycol, camellia sinensis (Green Tea) extract, cetrimonium bromide, cetyl alcohol, chondroitin sulfate, colloidal gold, colloidal trace mineral compounds, deionized water, dimethicone, DMDM hydantoin, ethoxydiglycol, ethylhexyl isononanoate, fragrance, glucosamine sulfate, glyceryl stearate, hydroxyethylcelluose, iodopropynyl butylcarbamate, lecithin, menthyl PCA, methyl-sulfonyl-methane (MSM), ordenone, PEG-100 stearate, phospholipids, polysorbate 60, PPG-12/SMDI copolymer, retinyl palmitate, rosmarinum officinalis (Rosemary) extract, saccharum officinarum (Sugar Cane) extract, salix alba (Willow) bark extract, stearyl alcohol, sodium carboxymethylcellulose, and tocopheryl acetate. In some embodiments, the methyl salicylate and menthol components are encapsulated by a protective membrane. In additional non-limiting embodiments, the pain-relieving composition comprises methyl salicylate from about 10%, menthol from about 10%, camphor from about 4%, and methyl nicotinate from about 0.25% by percentage weight.

Additionally, the present invention provides for a pain-relieving composition comprising lidocaine, menthol, camphor, deionized water, cetyl alcohol, ethylhexyl isononanoate, cetrimonium bromide, stearyl alcohol, glyceryl stearate, PEG-100 stearate, ethoxydiglycol, polysorbate 60, dimethicone, arnica Montana extract, arginine, methyl-sulfonyl-methane (MSM), glucosamine sulfate, chondroitin sulfate, colloidal gold, colloidal trace mineral compounds, tocopheryl acetate, retinyl palmitate, ascorbyl palmitate, phospholipids, lecithin, rosmarinum officinalis (Rosemary) extract, camellia sinensis (Green Tea) extract, salix alba (Willow) bark extract, menthyl PCA, hydroxyethylcelluose, sodium carboxymethylcellulose, PPG-12/SMDI copolymer, butylene glycol, ordenone, DMDM hydantoin, iodopropynyl butylcarbamate, and fragrance. In some embodiments, some or all of the components are encapsulated by a protective membrane. In preferred non-limiting embodiments, the pain-relieving composition comprises lidocaine from about 4.0%, menthol from about 1.0%, and camphor from about 3.0% by percentage weight.

Additionally, the present invention provides for a pain-relieving roll-on composition comprising menthol, methyl salicylate, camphor, capsaicin, SD alcohol, water, aloe vera, glucosamine, methylsulfonylmethane (MSM), colloidal gold, panax ginseng, glycerin, eugenia caryophyllus, cucumis sativus, aesculus hippocastanum, centella asiatica, rucus aculeatus, Symphytum officinale, salix alva, hypericum perforatum, glycyrrhizza glabra, panthenol B-5, hydrolized yeast protein, and hydrolyzed milk protein propylene glycol. Preferably, the pain-relieving composition comprises menthol from about 10.0%, methyl salicylate from about 8.5%, camphor from about 1.0%, and capsaicin from about 0.025% by percentage weight.

The present invention also provides for a pain-relieving spray composition comprising menthol, camphor, alcohol SD 40, propylene glycol, dimethyl sulfone, glucosamine HC1, peppermint oil, colloidal gold, arnica Montana extract, willow bark (salix alba) extract, green tea (camellia sinensis) extract, rosemary (Rosemarinus Officinalis) extract, FD&C Blue #1, and FD&C Yellow #5. Preferably, the spray composition comprises from about 10.0% Menthol and 3.0% Camphor by percentage weight. Facial Spray

In further non-limiting embodiments, the present invention provides for a facial spray composition further comprising at least one Protegenetic liposome. In some embodiments, the Protegenetic liposome comprises water, phosphatidylcholine, beta glucan, colloidal gold, EGF, copper PCA, rovisome SOD, ceramide II, sunflower oil, potassium sorbate, and sodium benzoate. In additional embodiments, the present invention provides for a Protegenetic liposome comprising water, phosphatidylcholine, squalane, retinyl palmitate, tocotrienols, rejuvenox, potassium sorbate, and sodium benzoate. In additional non-limiting embodiments, the facial spray composition further comprises at least two protegenetic liposomes comprising components according to the percentage by weight listed below:

| | |
|---|---|
| Protegenetic Liposome #1 Water | from about Q.S to 100% |
| Phosphatidylcholine | from about 10.0-20.0% |
| Beta Glucan | from about 0.5-3.25% |
| Colloidal Gold | from about 0.01-1.0% |
| EGF | from about 0.10-0.50% |
| Copper PCA | from about 0.15-0.35% |
| Rovisome SOD | from about 0.01-0.30% |
| Ceramide II | from about 0.20-0.40% |
| Sunflower Oil | from about 2.0-6.0% |
| Potassium Sorbate | from about 0.10-0.40% |
| Sodium Benzoate | from about 0.15-0.50% |
| Protegenetic Liposome #2 Water | from about Q.S. to 100% |
| Phosphatidylcholine | from about 10.0-20.0% |
| Squalane | from about 1.5-5.5% |
| Retinyl Palmitate | from about 0.01-0.30% |
| Tocotrienols | from about 0.15-0.45% |
| Rejuvenox | from about 0.10-0.60% |
| Potassium Sorbate | from about 0.10-0.40% |
| Sodium Benzoate | from about 0.15-0.50% |

In additional embodiments, the present invention provides for a facial spray composition comprising components according to the percentage by weight listed below:

| | |
|---|---|
| Water | from about QS to 100.00% |
| Sodium Hyaluronate | from about 0.50-2.50% |
| Transcutol | from about 1.00-4.00% |
| Sodium PCA | from about 1.50-4.00% |
| Colloidal Trace Minerals | from about 0.01-1.0% |
| Colloidal Gold | from about 0.01-1.0% |
| Protegenetic Liposome #1 | from about 2.0-9.00% |
| Protegenetic Liposome #2 | from about 2.0-9.00% |
| Potassium Sorbate | from about 0.01-0.30% |
| Sodium Benzoate | from about 0.15-0.50% |

Lubricant

The present invention provides for a silicone based lubricant composition comprising cyclopentasiloxane, dimethicone/vinyl dimethicone crosspolymer, arginine, colloidal gold, and gold leaf. Preferably, in some non-limiting embodiments, the composition comprises the components listed below according to the described percent weight ranges:

| | |
|---|---|
| Cyclopentasiloxane | from about QS to 100.00% |
| Dimethicone/Vinyl Dimethicone | from about 0.10-5.000% |
| Crosspolymer | |
| Arginine | from about 0.001-1.000% |
| Colloidal Gold | from about 0.001-10.00% |
| Gold Leaf | from about 0.001-1.000% |

Additionally, the present invention provides for a water based lubricant composition comprising water, propylene glycol, glycerin, carbomer, hydroxyethylcellulose, polyquaternium-7, diazolidinyl urea, methylparaben, propylparaben, colloidal gold, gold leaf, glyceryl acrylate/acrylic acid copolymer, propylene glycol, niacin, L-Arginine, panax ginseng root extract, and cinnamomum cassia bark extract. Preferably, in some non-limiting embodiments, the composition comprises the components listed below according to the described percent weight ranges:

| | |
|---|---|
| Water | from about QS to 100.00% |
| Propylene Glycol | from about 0.100-5.000% |
| Glycerin | from about 1.000-20.00% |
| Carbomer | from about 0.100-1.000% |
| Hydroxyethylcellulose | from about 0.100-1.000% |
| Polyquaternium-7 | from about 0.050-1.000% |
| Diazolidinyl Urea | from about 0.100-0.300% |
| Methylparaben | from about 0.050-0.300% |
| Propylparaben | from about 0.050-0.100% |
| Colloidal Gold | from about 0.001-5.000% |
| Gold leaf | from about 0.001-5.000% |
| Glyceryl Acrylate/Acrylic Acid Copolymer | from about 0.050-10.00% |
| Propylene Glycol | from about 0.100-20.00% |
| Niacin | from about 0.001-0.100% |
| L-Arginine | from about 0.001-1.000% |
| Panax Ginseng Root Extract | from about 0.001-5.000% |
| Cinnamomum Cassia Bark Extract | from about 0.001-1.000% |

Although described with specific examples, the present invention provides for some embodiments comprising variations of the components described above. Accordingly, variations, omissions, substitutions, and changes may be made by those skilled in the art without departing from the spirit of the present invention.

Methods

The present invention also relates to methods of administering any of the described compositions for the treatment and prevention of skin and/or integument conditions and diseases. As described above, administration may be carried out, for example, enterally, lingually, sublingually, topically, buccally, orally rectaliy, and/or parenterally.

By “administering” or “application,” it is meant that a composition is delivered to the subject in such a way that it can achieve the desired purpose. Compositions of the present invention can be administered to any suitable subject, including animals, humans, and other organisms. In the context of animals, the compositions of the present invention can also be used for veterinary administration to any suitable animal subject such as, for example, cats, dogs, or horses.

Generally, the treatment and prevention of skin or integument conditions involves contacting the metal containing composition with the area of skin, mucosal membrane, and/or integument having the condition or likely to have a condition. Such an area can be the site, or potential site, of an infection, inflammation, and/or other ailment. In some embodiments, the described composition is brought into contact with the site of infection via, for example, a medicated pad, shampoo, hair tonic, topical cream, transdermal patch, and/or wound dressing.

Additionally, in some embodiments, the present invention provides methods of treating a skin/integument condition by applying or administering an aerosol or spray to a site, or potential site, in need of treatment and prevention. In other embodiments, the described compositions are directly applied as powders, lotions, and/or sprays to a site of a wound, cut, laceration, or other trauma to the skin or integument. In the case of onychomycosis, some embodiments provide for applying the composition to the nail in an appropriate form such that the described composition penetrates the nail to contact the affected area.

According to some embodiments, the present invention provides for methods of administering the described composition enterally for the treatment and prevention of a skin/integument condition. These methods include use of tablets and/or other suitable carriers for the enteral administration to an animal or human in need of treatment and prevention.

In further embodiments, the present invention provides for methods of administering the described compositions buccally or orally. Oral or buccal administration may include oral hygiene formulations such as toothpastes, dental balms, or mouthwashes whereby the described compositions are brought into contact with the patient's mouth, gums, teeth, or cheeks.

In other embodiments, the present invention provides for parenteral administration of the described composition wherein the described composition is in a suitable solution. Parenteral administration may include injectable formulations whereby the described composition is administered intravenously to an animal or human for treatment or prevention of a skin or integument condition.

In further embodiments, the methods of the present invention provide for delivering the components of the composition via protective membrane structures such as liposomes, nanoshells, nanospheres, cerasomes, rovisomes, or nano-stmctures. In some embodiments, some or all of the described components of the composition are encapsulated by a protective membrane for delivery to the subject. In some embodiments, it is believed that the liposome encapsulated components fuse to the target cells and deliver the encapsulated contents through the cell membrane. In other embodiments, the contents can include proteins, enzymes, and other components for the beneficial healing, treatment and prevention of disease, and development of tissue.

In another embodiment, the present invention provides for methods of administering compositions with integument and skin-supporting components for the treatment and prevention of skin/integument conditions. These methods encompass modes of administration known to be suitable in the art, taking into consideration other factors like the needs of the subject treated.

Without wishing to be bound by any theory, it is believed that the novel compositions and methods of the present invention advantageously protect genes and their ability to produce hormones and enzymes by providing antigens, antioxidants, anti-aging agents, anti-wrinkle agents, anti-peroxidation agents, antimicrobial agents, anti-inflammatory agents, pain-relieving agents, wound recovery agents, sun-screens, sunblocks, and integument and skin-supporting agents. Moreover, it is believed, that the described compositions restore the natural organic balance of a youthful skin with transdermal hormone, enzyme, and peptide supplementation, allowing healthy genes to support natural regenesis.

Additionally, it is believed, that the described protective membrane encapsulated metal containing compositions bring antigens, antioxidants, and other nutritive supplements to the extracellular matrix where these can be the most effective for the regenerative process.

As will be appreciated now, the novel compositions and methods of the present invention are useful as antioxidants, anti-aging agents, anti-wrinkle agents, anti-peroxidation agents, antimicrobial agents, anti-inflammatory agents, pain-relieving agents, wound recovery agents, sun-screens, sunblocks, and integument and skin-supporting agents when applied to the skin/integument, or administered generally to an animal or human body. Additionally, the compositions and methods of the present invention will provide extensive application for treating and preventing skin and integument disorders, conditions, and diseases.

Although the foregoing description of the invention has shown, described and pointed out novel aspects of the invention, it will be understood that various omissions, substitutions, and changes in the composition as described, as well as the uses thereof, may be made by those skilled in the art without departing from the spirit of the present invention.

What is claimed is:

1. A composition, comprising colloidal metal and a cosmetically suitable carrier.

2. The composition according to claim 1, further comprising metal flakes.

3. The composition according to claim 1, wherein the colloidal metal comprises metal particles having a size from about 1-200 nm in a colloidal suspension.

4. The composition according to claim 2, wherein the metallic flakes have a particle size from about 1-5 mm.

5. The composition according to claim 1, wherein the colloidal metal comprises from about 0.001-50 percent by weight of the composition.

6. The composition according to claim 1, wherein the colloidal metal comprises a metal alloy.

7. The composition according to claim 2, wherein the metal flakes comprise a metal alloy.

8. The composition according to claim 1, wherein the colloidal metal is conjugated to a molecule.

9. The composition according to claim 8, wherein the conjugated colloidal metal comprises metal particles having a size from about 1-250 nm.

10. The composition according to claim 8, wherein the said molecule is selected from the group consisting of peptides, carbohydrates, enzymes, proteins, antigens, hormones, or polysaccharides.

11. The composition according to claim 1, wherein the suitable carrier comprises a skin-penetrating carrier.

12. The composition according to claim 11, wherein the said skin-penetrating carrier is selected from the group consisting of DMSO, liposomes, lipophilic solvents, lecithin, transcutol, nanospheres, nanoshells, or rovisomes.

13. The composition according to claim 11, wherein the said skin-penetrating carrier comprises a hollow and solid lipid structure.

14. The composition according to claim 11, wherein the said skin-penetrating carrier comprises a nano-structure.

15. The composition according to claim 1, further comprising of at least one additional agent.

16. The composition according to claim 15, wherein the said at least one additional agent is selected from the group consisting of a mineral, an emollient, an antimicrobial agent, an antioxidant, an analgesic, an anti-inflammatory agent, and a sebum-reducing agent.

17. The composition according to claim 15, wherein the at least one additional agent is selected from the group consisting of a hormone, an enzyme, a peptide, a protein, a lipid, a retinoid, a vitamin a wound recovery agent, a botanical extract, a MMP inhibitor, a polysaccharide, and an antigen.

18. The composition according to claim 15, wherein the said at least one additional agent comprises an integument and skin-supporting component.

19. A method of treating and/or preventing a skin/integument condition, comprising administering to a patient in need thereof, an amount of the composition of claim 1, wherein the amount is sufficient to treat and/or prevent the skin/integument condition.

20. The method according to claim 19, wherein the mode of administration is selected from the group consisting of topical, oral, transdermal, mucosal, enteral, parenteral, or sublingual administration.
